(19) United States
(12) Patent Application Publication
(10) Pub. No.: US 2014/0073528 A1
(43) Pub. Date: Mar. 13, 2014

(54) METHODS OF DEPLETING A TARGET MOLECULE IN A SAMPLE AND KITS FOR PRACTICING THE SAME

(71) Applicant: Clontech Laboratories, Inc., Mountain View, CA (US)

(72) Inventors: Craig Betts, Mountain View, CA (US); Eric Espinosa, Mountain View, CA (US); Magnolia Bostick, Mountain View, CA (US)

(21) Appl. No.: 14/023,358

(22) Filed: Sep. 10, 2013

Related U.S. Application Data
(60) Provisional application No. 61/700,681, filed on Sep. 13, 2012.

(51) Int. Cl. C12N 15/10

(52) U.S. Cl. CPC C12N 15/1003
USPC 506/9; 435/6.12

(57) ABSTRACT

Provided are methods of depleting a target molecule in a sample. The methods include contacting a target molecule with a free radical-generating system and generating free radicals from the free radical-generating system to deplete the target molecule in the sample. Kits for practicing the subject methods are also provided.

### CROSS REFERENCE TO RELATED APPLICATIONS

Pursuant to 35 U.S.C. §119 (e), this application claims priority to the filing date of the U.S. Provisional Patent Application Ser. No. 61/700,681, filed Sep. 13, 2012; the disclosure of which is herein incorporated by reference.

### INTRODUCTION

Applications in biomedical research often involve the analysis of specific subsets of nucleic acids present in a complex mixture of other sequences—for example, analysis of gene expression by array hybridization, qPCR or massively parallel sequencing. If the target sequences are known, PCR with specific primer sequences can be used to amplify the desired sequences out of the mixture. In some cases, however, it may be desired to analyze multiple different sequences, perhaps where sequence information is not fully known. Messenger RNAs in eukaryotic systems, for example, may be collectively amplified and analyzed using an oligo-dT primer to initiate first strand cDNA synthesis by priming on the poly A tail, thereby reducing or avoiding contamination by unwanted nucleic acids—such as ribosomal RNAs, mitochondrial RNAs and genomic DNA. A requirement for this approach, however, is that the RNA is intact and not degraded, e.g., the poly A tails are not lost or disconnected from the body of the RNA message. Unfortunately, many otherwise useful and interesting biological specimens—such as biopsied material retained as formalin-fixed and paraffin embedded tissue samples (FFPE samples) often suffer from such degradation making oligo-dT priming impractical for such samples. Further, many interesting RNA sequences do not have poly A tails—e.g., non-coding RNAs and non-eukaryotic RNAs. In such cases, random priming can be used to generally amplify all nucleotide species in the sample. However, random priming will also result in the amplification of potentially unwanted sequences—such as genomic DNA or ribosomal RNA.

### SUMMARY

Provided are methods of depleting a target molecule in a sample. The methods include contacting a target molecule with a free radical-generating system and generating free radicals from the free radical-generating system to deplete the target molecule in the sample. Kits for practicing the subject methods are also provided.

### BRIEF DESCRIPTION OF THE FIGURES

FIG. 1 schematically illustrates a method according to one embodiment of the present disclosure.

### DETAILED DESCRIPTION

Provided are methods of depleting a target molecule in a sample. The methods include contacting a target molecule with a free radical-generating system and generating free radicals from the free radical-generating system to deplete the target molecule in the sample. Kits for practicing the subject methods are also provided.

Before the methods and kits of the present disclosure are described in greater detail, it is to be understood that the methods and kits are not limited to particular embodiments described, as such may, of course, vary. It is also to be understood that the terminology used herein is for the purpose of describing particular embodiments only, and is not intended to be limiting, since the scope of the methods and kits will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods and kits. The upper and lower limits of these smaller ranges may independently be included in the smaller ranges and are also encompassed within the methods and kits, subject to any specifically excluded limit in the stated range. Where the stated range includes one or both of the limits, ranges excluding either or both of those included limits are also included in the methods and kits.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods belong. Although any methods and kits similar or equivalent to those described herein can also be used in the practice or testing of the methods and kits, representative illustrative methods and materials are now described.

All publications and patents cited in this specification are herein incorporated by reference as if each individual publication or patent were specifically and individually indicated to be incorporated by reference and are incorporated herein by reference to disclose and describe the methods, kits and/or materials in connection with which the publications are cited. The citation of any publication is for its disclosure prior to the filing date and should not be construed as an admission that the present methods and kits are not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods and kits, which are, for clarity, described in the context of separate embodiments, may also be provided in combination in a single embodiment. Conversely, various features of the methods and kits, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed, to the extent that such combinations embrace operable processes and/or devices/systems/kits. In addition, all sub-combinations listed in the embodiments describing such variables are also specifically embraced by the present methods and kits and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods and kits. Any recited method can be carried out in the order of events recited or in any other order which is logically possible.

In further describing embodiments of the invention, aspects of embodiments of the subject methods will be described first in greater detail. Thereafter, aspects of embodiments of the kits for practicing the subject methods are described in greater detail.

### Methods

Embodiments of the present disclosure relate to methods of depleting a target molecule in a sample. The methods include contacting a target molecule with a free radical-generating system and generating free radicals from the free radical-generating system to deplete the target molecule in the sample.

Target Molecules

The subject methods find use in depleting a wide variety of target molecules. The target molecule may be any type of molecule (or sub-type thereof) that a practitioner of the subject methods desires to deplete in the sample. Target molecules which may be depleted using the subject methods include, but are not limited to, nucleic acids, polypeptides, carbohydrates (e.g., saccharides, such as oligosaccharides, polysaccharides, etc.), and combinations thereof.

In certain aspects, the target molecule is a nucleic acid (i.e., a “target nucleic acid”). By “nucleic acid” is meant a polymer of any length, e.g., greater than about 10 bases, greater than about 20 bases, greater than about 50 bases, greater than about 100 bases, greater than about 500 bases, greater than about 1000 bases, greater than about 2000 bases, greater than about 3000 bases, greater than about 4000 bases, greater than about 5000 bases, greater than 10,000 bases, up to about 50,000 or more bases composed of nucleotides, e.g., ribonucleotides or deoxyribonucleotides.

By “depleting a target nucleic acid,” it is meant reducing the percentage of a type of undesired nucleic acid (e.g., ribosomal RNA (rRNA) or one or more particular sub-types thereof) in a sample with respect to the total nucleic acid in the sample. In certain aspects of the present disclosure, after depletion of the target nucleic acid, the percent remaining of the target nucleic acid as compared to the initial amount of target nucleic acid in the sample is 20%, 19%, 18%, 17%, 16%, 15%, 14%, 13%, 12%, 11%, 10%, 9%, 8%, 7%, 6%, 5%, 4%, 3%, 2%, 1%, or less, including 0.5%, 0.1%, 0.01% or less. By depleting a target nucleic acid in a sample, a desired type of nucleic acid (e.g., messenger RNA (mRNA), micro RNA (miRNA), and/or any other desired type of nucleic acid) may be enriched. According to certain embodiments, in a sample in which the target nucleic acid has been depleted, a desired type of nucleic acid is enriched such that the amount of the desired type of nucleic acid relative to the total nucleic acid in the samples increases by 5% or more, such as 10% or more, 25% or more, 30% or more, 40% or more, 50% or more, 60% or more, 70% or more, including 80%, 81%, 82%, 83%, 84%, 85%, 86%, 87%, 88%, 89%, 90%, 91%, 92%, 93%, 94%, 95%, 96%, 97%, 98%, 99%, and 99.5% or more.

The nucleic acid targeted for depletion can be any target nucleic acid selected by a practitioner of the subject methods. According to one embodiment, the target nucleic acid is a ribonucleic acid (RNA). The RNA targeted for depletion may be any type of RNA (or sub-type thereof) including, but not limited to, a ribosomal RNA (rRNA), a microRNA (miRNA), a messenger RNA (mRNA), transfer RNA (tRNA), a small nucleolar RNA (snoRNA), a small nuclear RNA (snRNA), a long non-coding RNA (IncRNA), a non-coding RNA (ncRNA), a small interfering RNA (siRNA), a transacting small interfering RNA (ta-siRNA), a natural small interfering RNA (nat-siRNA), a transfer-messenger RNA (tmRNA), a precursor messenger RNA (pre-mRNA), a small Cajal body-specific RNA (scaRNA), a piwi-interacting RNA (piRNA), an endoribonuclease-prepared siRNA (esiRNA), a small temporal RNA (sRNA), a signal recognition RNA, a telomere RNA, a ribozyme, and any combination of RNA types thereof or subtypes thereof. When the target nucleic acid is a RNA, the methods may include depleting all types of RNA in the sample (e.g., ribosomal RNA, transfer RNA, microRNA, and the like), or one or more particular types of RNA. In certain aspects, the target nucleic acid is ribosomal RNA (rRNA). The rRNA may be a eukaryotic 28S, 26S, 25S, 18S, 5.8S, 5S rRNA, or any combination thereof. In other aspects, the rRNA may be a prokaryotic 23S, 16S, 5S rRNA, or any combination thereof. The subject methods find use in depleting RNAs other than ribosomal RNAs. For example, the target nucleic acid may be a messenger RNA (mRNA), e.g., a highly expressed but clinically irrelevant mRNA from a pool of total RNA or mRNA (e.g., a globulin mRNA in a sample of total or polyA⁺ blood RNA). Other types of RNA may be targeted for depletion, including a precursor messenger RNA (pre-mRNA), a micro RNA (miRNA), a transfer RNA (tRNA), a mitochondrial RNA (mtRNA), and any combination thereof. The target RNA may be a RNA from a particular organism, such as bacterial RNA or yeast RNA.

According to certain embodiments, the target molecule is a target nucleic acid, and the target nucleic acid is a deoxyribonucleic acid (DNA), e.g., intronic or inter-geneic DNA when it is desired to enrich a sample for exonic DNA. In certain aspects, DNA-based plasmids/vectors such as those used for in vitro transcription may be targeted for depletion, e.g., after completion of an in vitro transcription reaction to enrich a nucleic acid sample for newly transcribed RNA.

The term “sample”, as used herein, relates to a material or mixture of materials, typically, although not necessarily, in liquid form, containing nucleic acids and/or proteins which one desires to deplete. A sample may be derived from any source of interest. For example, the sample may be a mixture of undesired and desired nucleic acids and/or undesired and desired proteins isolated from a single cell, a plurality of cells, a tissue, an organ, or an organism(s) (e.g., bacteria, yeast, or the like). Approaches, reagents and kits for isolating DNA, RNA, and/or protein from such sources are known in the art. For example, kits for isolating total RNA from cells or tissues of interest—such as the NucleoSpin® RNA kits by Clontech Laboratories, Inc. (Mountain View, Calif.)—are commercially available. In certain aspects, the sample is a mixture of undesired and desired nucleic acids isolated from fixed tissue, e.g., formalin-fixed, paraffin-embedded (FFPE) tissue. Total RNA from FFPE tissue may be isolated using commercially available kits—such as the NucleoSpin® FFPE RNA kits by Clontech Laboratories, Inc. (Mountain View, Calif.). Kits for isolating polyA⁺ mRNA from total RNA or directly from cells or tissue—such as the NucleoTrap® mRNA kits by Clontech Laboratories, Inc. (Mountain View, Calif.)—are commercially available. Small RNAs such as miRNAs, siRNAs, shRNAs and snRNAs may be isolated from cells, tissues or plasma using commercially available kits—such as the NucleoSpin® miRNA kits by Clontech Laboratories, Inc. (Mountain View, Calif.).

In certain embodiments, the target molecule is a polypeptide (i.e., a “target polypeptide”). By target polypeptide is meant a polymer of any length, e.g., 10 residues or longer, 20 residues or longer, 25 residues or longer, 50 residues or longer, 100 residues or longer, 200 residues or longer, 500 residues or longer, 1000 residues or longer, 2500 residues or longer, etc.

A polypeptide targeted for depletion can be any target polypeptide selected for depletion by a practitioner of the subject methods. According to one embodiment, the target polypeptide is a protein (e.g., an enzyme) capable of degrading a desired nucleic acid (e.g., DNA and/or RNA) or protein in a sample. For example, when it is desirable to minimize degradation of RNA in a sample, the polypeptide targeted for depletion may be a ribonuclease (e.g., RNase A). Similarly, when it is desirable to minimize degradation of DNA in a sample, the polypeptide targeted for depletion may be one or more nucleases that degrade DNA (e.g., DNase I). When it is desirable to minimize degradation of protein in a sample, the polypeptide targeted for depletion may be one or more proteases and/or peptidases including, but not limited to: serine proteases, metalloproteases, cysteine proteases, aminopeptidases, aspartic proteases, and combinations thereof. Target polypeptides also include proteins that interfere with downstream applications of interest, such as albumin in cell culture supernatants. In other aspects, the polypeptide targeted for depletion is a polypeptide for which one would like to elucidate the activity/function of the polypeptide. For example, a particular protein could be depleted in a sample (e.g., a cell extract, etc.), and one could then determine the activity/function of that protein by determining what activity is lost in the sample, as compared to a sample in which that protein was not depleted.

Free Radical-Generating Systems

In practicing the subject methods, the target molecule is contacted with a free radical-generating system. By “free radical-generating system” is meant any collection of molecular and/or non-molecular components capable of generating free radicals sufficient to deplete the target molecule, e.g., by cleaving the target molecule, modifying (e.g., inactivating) the target molecule (e.g., by generating target molecule adducts), and/or the like.

The free radical-generating system may be provided in the sample at any final concentration suitable for the desired extent of target molecule depletion. For example, the final concentration the free radical-generating system may range from 1 nM to 10 mM, such as 1 μM to 1 mM.

in certain aspects, the target molecule is contacted with a single free radical-generating system. Alternatively, the target nucleic acid may be contacted with 2, 3, 4, 5, 6, 7, 8, 9, 10, 15, 20, 30, 40, 50, 100, 500, or 1000 or more free radical-generating systems. One practicing the subject methods may select the number of free radical-generating systems based on the size and number of different types of target molecules to be depleted (if one desires to deplete more than one type of target molecule in the sample) and the desired extent of target molecule depletion (e.g., cleavage)—where the number of free radical-generating systems that contact the target molecule (e.g., at distinct regions of the target molecule) may be increased to increase the extent of depletion. Free radical-generating systems may be used to specifically deplete one particular target molecule (e.g., a eukaryotic 28S, 18S, 5.8S, or 5S ribosomal RNA), or two or more different target molecules (e.g., any combination of two or more eukaryotic ribosomal RNAs selected from 28S, 18S, 5.8S, and 5S ribosomal RNA). For example, the subject methods can be used to concurrently deplete the 28S and 18S populations of eukaryotic rRNAs in a sample using a single species of free radical-generating system or, alternatively, a cocktail of different species of free radical-generating systems configured to deplete two or more different target molecules in the sample.

In certain aspects, the free radical-generating system includes a catalyst (e.g., a metal such as iron or copper). The system may include a catalyst alone, or the system may include a catalyst and a catalyst binding agent. When the free radical-generating system includes a catalyst binding agent, the catalyst binding agent may include a targeting molecule that binds (e.g., specifically binds) to the target molecule. These and other example components of free radical-generating systems are described in detail herein below.

Catalysts and Catalyst-Binding Agents

As set forth above, in certain aspects, the free radical-generating system includes a catalyst. The catalyst may be employed to catalyze a reaction that generates free radicals. In certain aspects, the catalyst for generating free radicals is a metal (e.g., a transition metal). For example, the catalyst may be a metal selected from copper (Cu), iron (Fe), and nickel (Ni). Any metal capable of catalyzing a reaction that generates the desired type of free radical may be used.

When the free radical-generating system includes a catalyst, the system may further include a catalyst-binding agent. One practicing the subject methods can select a catalyst-binding agent and catalyst according to the desired type of free radical to be generated for depletion of the target polymer, the type of targeting molecule to which the catalyst binding agent is to be attached, and the like.

In certain aspects, the catalyst-binding agent and catalyst form a chelate in which the catalyst-binding agent is a chelating agent and the catalyst is a metal ion. The chelating agent may be selected from ethylenediaminetetraacetic acid (EDTA), derivatives of EDTA (e.g., p-bromoacetamidobenzyl-EDTA (BABE), EDTA-2-aminoethyl-2-pyridyldisulfide (EPD), or any other EDTA derivative), 1,10-phenanthroline, derivatives of 1,10-phenanthroline (e.g., 5-iodoacetamido-1,10-phenanthroline (loP), or any other 1,10-phenanthroline derivative), a hydroxamate moiety or linkage, nitrilotriacetic acid (NTA) or any derivative of NTA, iminodiacetic acid (IDA) or any derivative of IDA, carboxymethylaspartate (CMA) or any derivative of CMA, ethylene glycol tetraacetic acid (EGTA) or any derivative of EGTA, porphyrin, combinations thereof, and any other chelating agent capable of forming a chelate with the desired metal ion. According to one embodiment, the chelating agent is EDTA or a derivative thereof and the metal ion is iron (e.g., ferrous iron (Fe²⁺)). In other aspects, the chelating agent is 1,10-phenanthroline or a derivative thereof and the metal ion is copper (Cu²⁺).

Targeting Molecules

According to certain aspects, when the subject methods employ a free radical-generating system that includes a catalyst and catalyst-binding agent, the catalyst-binding agent may include a targeting molecule. By “targeting molecule” is meant a molecule attached to (or otherwise associated with) the catalyst-binding agent, which molecule brings the catalyst-binding agent (and accordingly, the catalyst) into close proximity of the target molecule. By “close proximity” is meant the catalyst-binding agent and/or catalyst is brought within a radius of the target molecule of less than 100 Å, less than 90 Å, less than 80 Å, less than 70 Å, less than 60 Å, less than 50 Å, less than 40 Å, less than 30 Å, less than 20 Å, less than 10 Å, or less than 5 Å. Any targeting molecule capable of bringing the catalyst-binding agent into close proximity of the target molecule may be used.

Suitable targeting molecules may be selected based upon the type of target molecule to be depleted. As described above, in certain aspects, the target molecule is a nucleic acid (e.g., an rRNA, or any other nucleic acid). When the target molecule is a nucleic acid, any targeting molecule capable of bringing the catalyst-binding agent into close proximity of the target nucleic acid may be used. Example targeting molecules that may be used when the target molecule is a nucleic acid include, but are not limited to, a nucleic acid that specifically hybridizes via a complementary sequence to the target nucleic acid, a nucleic acid that targets the target nucleic acid by strand invasion, a polypeptide (e.g., a polypeptide that binds (e.g., specifically binds) to the target nucleic acid), an intercalating agent that intercalates the target nucleic acid (e.g., only double-stranded nucleic acids), and an aptamer (e.g., an aptamer that binds to the target nucleic acid).

As set forth above, when the target molecule is a nucleic acid, an example targeting molecule is a nucleic acid (i.e., a “targeting nucleic acid”) that specifically hybridizes to the target nucleic acid. By “specifically hybridizes” is meant the ability of the targeting nucleic acid to hybridize to all or a portion of the target nucleic acid. In some instances, the targeting nucleic acid hybridizes, if at all, to non-target nucleic acids only partially or weakly and in some instances the targeting nucleic acid does not hybridize to any nucleic acid other than to the target nucleic acid. Whether the targeting nucleic acid specifically hybridizes to a target nucleic acid is determined by such factors as the degree of complementarity between the targeting nucleic acid and the target nucleic acid and the temperature at which the hybridization occurs, which may be informed by the melting temperature (T_{M}) of the targeting nucleic acid. The melting temperature refers to the temperature at which half of the targeting nucleic acid-target nucleic acid duplexes remain hybridized and half of the duplexes dissociate into single strands. The Tₘ of a duplex may be experimentally determined or predicted using the following formula Tₘ=81.5+16.6(log₁₀[Na⁺])+0.41 (fraction G+C)−(60/N), where N is the chain length and [Na⁺] is less than 1 M. See Sambrook and Russell (2001; Molecular Cloning: A Laboratory Manual, 3^{rd} ed., Cold Spring Harbor Press, Cold Spring Harbor N.Y., Ch. 10). Other more advanced models that depend on various parameters may also be used to predict Tₘ of duplexes may also be used depending on various hybridization conditions. Approaches for achieving specific nucleic acid hybridization may be found in, e.g., Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier (1993). In certain aspects, the targeting molecule is a targeting nucleic acid selected from a DNA, an RNA, a locked nucleic acid (LNA), a peptide nucleic acid (PNA), and a xeno nucleic acid (XNA).

The term “complementary” as used herein refers to a targeting nucleic acid sequence that base-pairs by non-covalent bonds to a target nucleic acid. In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. The term “complementary” may also encompass duplexes that are fully complementary such that every nucleotide in one strand is complementary to every nucleotide in the other strand in corresponding positions. In certain cases, a nucleotide sequence may be partially complementary to a target, in which not all nucleotide is complementary to every nucleotide in the target nucleic acid in all the corresponding positions. For example, the targeting nucleic acid may be perfectly (i.e., 100%) complementary to the target nucleic acid, or the targeting nucleic acid and the target nucleic acid may share some degree of complementarity which is less than perfect (e.g., 70%, 75%, 85%, 90%, 95%, 99%). The percent identity of two nucleotide sequences can be determined by aligning the sequences for optimal comparison purposes (e.g., gaps can be introduced in the sequence of a first sequence for optimal alignment). The nucleotides at corresponding positions are then compared, and the percent identity between the two sequences is a function of the number of identical positions shared by the sequences (i.e., (% identity=# of identical positions/total # of positions×100). When a position in one sequence is occupied by the same nucleotide as the corresponding position in the other sequence, then the molecules are identical at that position. A preferred, non-limiting example of such a mathematical algorithm is described in Karlin et al., Proc. Natl. Acad. Sci. USA 90:5873-5877 (1993). Such an algorithm is incorporated into the NBLAST and XBLAST programs (version 2.0) as described in Altschul et al., Nucleic Acids Res. 25:389-3402 (1997). When utilizing BLAST and Gapped BLAST programs, the default parameters of the respective programs (e.g., NBLAST) can be used. In one aspect, parameters for sequence comparison can be set at score=100, wordlength=12, or can be varied (e.g., wordlength=5 or wordlength=20).

Generating a targeting nucleic acid suitable for practicing the subject methods may include identifying a unique region of the target nucleic acid and synthesizing a targeting nucleic acid that is complementary (e.g., perfectly complementary) to the unique region. By “unique region” is meant a nucleic acid sequence within the target nucleic acid that is not present in any other nucleic acids in the sample of interest. In certain aspects, the unique region is a region in a ribosomal RNA (e.g., a eukaryotic 28S, 26S, 25S, 18S, 5.8S, or 5S rRNA). Sequence analysis (e.g., a BLAST search) may be performed to determine which regions of a target nucleic acid are unique with respect to the sample of interest. Once the unique regions are identified, a targeting nucleic acid of sufficient complementarity, length and/or G:C content may be synthesized such that the targeting nucleic acid specifically hybridizes to a unique region.

According to one embodiment, the target molecule is a target nucleic acid, and the targeting molecule is an oligonucleotide that specifically hybridizes to the target nucleic acid. By “oligonucleotide” is meant a single-stranded multimer of nucleotides from 2 to 500 nucleotides, e.g., 2 to 200 nucleotides. Oligonucleotides may be synthetic or may be made enzymatically, and, in some embodiments, are under 10 to 50 nucleotides in length. Oligonucleotides may contain ribonucleotide monomers (i.e., may be oligoribonucleotides or “RNA oligonucleotides”) or deoxyribonucleotide monomers (i.e., may be oligodeoxyribonucleotides or “DNA oligonucleotides”). Oligonucleotides may be 10 to 20, 11 to 30, 31 to 40, 41 to 50, 51-60, 61 to 70, 71 to 80, 80 to 100, 100 to 150 or 150 to 200, up to 500 or more nucleotides in length, for example.

As set forth above, when the target molecule is a nucleic acid, an example targeting molecule is a polypeptide (i.e., a “targeting polypeptide”). In certain aspects, the targeting polypeptide specifically binds to a nucleotide sequence (e.g., a recognition sequence) in the target nucleic acid. In this context, suitable targeting molecules include nucleic acid binding proteins (e.g., proteins having a zinc finger, a helix-turn-helix, a leucine zipper, or other nucleic acid binding domain) such as transcription factors, endonucleases, or modified variants thereof capable of binding to a sequence within the target nucleic acid. In certain aspects, when the target molecule is a target nucleic acid, the target nucleic acid is a ribosomal RNA and the targeting molecule is a ribosomal protein or binding fragment thereof, e.g., a protein found in a small or large subunit in eukaryotic or prokaryotic ribosomes. Ribosomal proteins from various eukaryotes, prokaryotes, and archae that find use as targeting molecules for depleting rRNA include those described in the Ribosomal Protein Gene Database (RPG) (available on the world wide web at ribosome.med.miyazaki-u.ac.jp).

As described above, when the target molecule is a nucleic acid, an example targeting molecule is an intercalating agent. Intercalating agents that find use as targeting molecules in the subject methods include, but are not limited to, berberine, acridines such as proflavine and quinacrine, daunomycin, doxorubicin, thalidomide, aflotoxin B1, and ethidium bromide.

Another example targeting molecule that may be used when the target molecule is a nucleic acid is an aptamer. Aptamers are oligonucleotides (e.g., short DNA, RNA, XNA, or other short nucleic acid molecules) or peptides selected from random pools based on their ability to bind molecules including nucleic acids, proteins, and small organic compounds. Aptamers that find use in targeting a free radical generating system to the target molecule (e.g., a target nucleic acid, a target protein, or any other target molecule) may be identified and/or engineered using approaches such as repeated rounds of “in vitro selection” or “SELEX” (systematic evolution of ligands by exponential enrichment).

Depletion of a target nucleic acid may produce a fragmented target nucleic acid, the fragments of which may be removed from the sample if desired. In certain embodiments, a target nucleic acid (e.g., an rRNA) is contacted with multiple different free radical generating systems at distinct regions of the target nucleic acid such that all or nearly all of the target nucleic acid fragments are sufficiently small to be removed by nucleic acid purification steps such as ethanol or isopropanol precipitation, spin column purification (e.g., using NucleoSpin® Clean-Up columns by Clontech Laboratories, Inc. (Mountain View, Calif.)), or the like.

The subject methods may further include deactivating and/or removing the free radical generating system from the sample, e.g., once free radical generation has occurred for a desired period of time. For example, when the targeting molecule of the free radical generating system is a nucleic acid, the free radical generating system may be deactivated by contacting the targeting nucleic acid with a nuclease that degrades the targeting nucleic acid. Other approaches for removing a free radical generating system that includes a nucleic acid targeting molecule may be employed. According to one embodiment, the nucleic acid targeting molecule includes one or more nucleotides that are not normally present in a DNA or RNA molecule, where the nucleotide not normally present marks the nucleic acid targeting molecule for cleavage and/or degradation when contacted with a particular enzyme. For example, the triphosphate form of deoxyuridine (dUTP) is present in living organisms as a metabolic intermediate, but it is rarely incorporated into DNA. When dUTP is incorporated into DNA, the resulting deoxyuridine is promptly removed in vivo, e.g. by processes involving the enzyme uracil-N-glycosylase (UDG). In certain aspects, the subject methods employ a DNA-based targeting molecule in which dUTP is incorporated. Following the free radical generation step of the subject methods, the uracil-containing oligonucleotides may be contacted with UDG, which cleaves the glycosidic bond between the deoxyribose of the DNA sugar-phosphate backbone and the uracil base, effectively degrading the DNA-based targeting molecule and/or facilitating its subsequent removal by alcohol precipitation, spin column purification, or other suitable removal strategies.

When the target molecule is a polypeptide, any targeting molecule capable of bringing the catalyst-binding agent into close proximity of the target polypeptide may be used. Example targeting molecules that may be used when the target molecule is a polypeptide include, but are not limited to, an antibody that specifically binds to the target polypeptide, a polypeptide that is a natural binding partner with the target polypeptide, a ligand that binds to the target polypeptide, a small molecule that binds to the target polypeptide, a nucleic acid (e.g., a nucleic acid that includes a target polypeptide recognition sequence), an aptamer (e.g., an aptamer that binds (e.g., specifically binds) to the target polypeptide).

As set forth above, when the target molecule is a target polypeptide, the targeting molecule may be any molecule (e.g., an antibody, an aptamer, a ligand or the like) capable of specifically binding to the target polypeptide. In certain embodiments, the affinity between a targeting molecule and a target polypeptide to which it specifically binds when they are specifically bound to each other in a binding complex is characterized by a K_{D} (dissociation constant) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. A variety of different types of specific binding agents may be employed as the targeting molecules. Specific binding agents of interest include antibody binding agents, aptamers, ligands, proteins, peptides, haptens, nucleic acids, etc. The term “antibody binding agent” as used herein includes polyclonal or monoclonal antibodies or fragments thereof that are sufficient to bind to the target polypeptide. The antibody fragments can be, for example, monomeric Fab fragments, monomeric Fab′ fragments, or dimeric F(ab)′₂ fragments. Also within the scope of the term “antibody binding agent” are molecules produced by antibody engineering, such as single-chain antibody molecules (scFv) or humanized or chimeric antibodies produced from monoclonal antibodies by replacement of the constant regions of the heavy and light chains to produce chimeric antibodies or replacement of both the constant regions and the framework portions of the variable regions to produce humanized antibodies.

Attachment of Targeting Molecules to Catalyst-Binding Agents

As described above, in certain aspects, free radical-generating systems that include a catalyst and catalyst-binding agent may employ a catalyst-binding agent that includes a targeting molecule that brings the catalyst-binding agent into close proximity of the target molecule. The catalyst-binding agent may include a targeting molecule by being covalently or non-covalently bound to the targeting molecule, so long as the catalyst-binding agent and targeting molecule are stably associated under the conditions of use. In certain aspects, the catalyst-binding agent is covalently attached to the targeting molecule. Suitable approaches for attaching the catalyst-binding agent to a targeting molecule will vary depending on the type of catalyst-binding agent and targeting molecule selected by a practitioner of the subject methods. Chelates such as EDTA-Fe(II), porphyrin-Fe(II), 1,10-phenanthroline-Cu(II), and BABE-Fe(II), for example, have been successfully tethered to nucleic acids (e.g., oligonucleotides) and proteins.

According to certain embodiments, the targeting molecule is a nucleic acid that binds (e.g., specifically hybridizes, specifically binds, etc.) to a target molecule (e.g., a target nucleic acid or a target polypeptide), and the catalyst-binding agent is covalently attached to the targeting nucleic acid. Strategies for chemically attaching chelating agents (with or without bound metal ions) to nucleic acids are described in, e.g., Bowen et al. (Methods (2001) 25:344-350), Chen and Sigman (PNAS (1986) 83:7147-7151), Francois et al. (Nucleic Acids and Molecular Biology (2004) 13:223-242), Hermann and Neumann (Methods Enzymol. (2000) 318:33-43), Joseph and Noller (Methods Enzymol. (2000) 318:175-190) and Muth and Hill (Methods (2001) 23:218-232), the disclosures of which are incorporated herein by reference in their entireties for all purposes.

Linkages that find use in attaching a catalyst-binding agent to a targeting nucleic acid include phosphorothioate linkages, hydroxamate linkages, and any other linkages compatible with the particular combination of catalyst-binding agent and targeting nucleic acid used to make the free radical-generating system. In certain aspects, the target molecule is a target nucleic acid, and the free radical-generating system includes a targeting nucleic acid derivatized with EDTA-Fe(II) or 1,10-phenanthroline-Cu(II) via a phosphorothioate linkage. A targeting nucleic derivatized in this manner may be generated by performing the following steps. First, a targeting nucleic acid (e.g., an oligonucleotide) complementary to the target nucleic acid may be synthesized using phosphoramidate chemistry to include a 5′-phosphorothioate by use of a 5′-phosphorylating reagent during the last step of the synthesis. In the case of EDTA-Fe(II), the EDTA derivative BABE may be loaded with Fe²⁺ by mixing BABE in DMSO with fresh Fe²⁺ (NH₄⁺)₂(SO₄²⁻)₂.6H₂O and incubating at room temperature for 60 minutes. Attachment of Fe(II)-BABE to the 5′-phosphorothioate on the targeting nucleic acid may be accomplished by mixing purified 5′-phosphorothioate targeting nucleic acid with a lyophilized Fe(II)-BABE pellet generated above, potassium phosphate buffer (pH 8.5) and water. This mixture may be incubated at 37° for 2 hours, followed by the addition of EDTA to chelate any remaining uncomplexed Fe²⁺. The resulting Fe(II)-BABE-targeting nucleic acid complex may be purified by phenol-chloroform extraction followed by ethanol precipitation and resuspension in water for subsequent use as a free radical-generating system in the subject methods. Similarly, in the case of 1,10-phenanthroline-Cu(II), the derivative 5-iodoacetamido-1,10-phenanthroline (loP) in DMSO may be added to 5′-phosphorothioate targeting nucleic acid (generated as described above) in a 1:1 DMSO:water solution and incubated at room temperature for 60 minutes to generate a Cu(II)-loP-targeting nucleic acid complex which upon purification may be used as a free radical-generating system in the subject methods. Further details regarding the generation of a 5′-phosphorothioate targeting nucleic acids and attachment of Fe(II)-BABE or Cu(II)-loP thereto may be found, e.g., in Bowen et al. (Methods (2001) 25:344-350).

As set forth above, in certain aspects, the catalyst-binding agent may be attached to a targeting nucleic acid via a hydroxamate linkage, where hydroxamate linkages in nucleic acids have been shown to effectively chelate iron when there are three non-adjacent internucleosidic hydroxamate linkages available in a single nucleic acid. Synthetic nucleic acids that include hydroxamate linkages may be generated by first converting the 5′-hydroxyl group of a nucleoside into the corresponding 5′-N-hydroxylamino substituted derivative that includes the hydroxamate linkage by reacting the nucleoside with N-(tert-butoxycarbonyl)-O-(benzyloxycarbonyl)hydroxylamine (BocNHOCbz). Synthesizing the targeting nucleic acid complementary to the target nucleic acid may be performed by incorporating 5′-N-hydroxylamino substituted nucleotides (which include the hydroxamate linkage) at selected positions of the targeting nucleic acid via solid phase synthesis. Further details regarding the generation of hydroxamate linkage-containing nucleic acids may be found, e.g., in Miller et al. (Biometals (2009) 22:491-510).

According to certain embodiments, the targeting molecule is a polypeptide that binds (e.g., specifically binds) to a target molecule (e.g., a polypeptide, ligand, nucleic acid, or other target molecule), and the catalyst-binding agent is covalently attached to the targeting polypeptide. Strategies for chemically attaching chelating agents to polypeptides are described in, e.g., Heilek et al. (PNAS (1995) 92:1113-1116), Heilek and Noller (RNA (1996) 2:597-602), Hall and Fox (Methods (1999) 18:78-84), Wilson and Noller (Cell (1998) 92:131-139), and Beck et al. (RNA (1998) 4:331-339), the disclosures of which are incorporated herein by reference in their entireties for all purposes.

Linkages that find use in attaching a catalyst-binding agent to a targeting nucleic acid include, but are not limited to, disulfide linkages. Disulfide linkages may be provided, e.g., at naturally-occurring or genetically engineered cysteine residues of the targeting polypeptide. In certain aspects, the targeting molecule is a protein (e.g., an antibody, ligand, or the like) that includes one or more naturally-occurring and/or genetically-engineered cysteines (e.g., by site-directed mutagenesis). According to certain embodiments, a free thiol group (S—H) of a cysteine-containing targeting polypeptide is reacted with a free thiol group of a catalyst-binding agent. For example, a thiol group of the targeting polypeptide may be reacted with a thiol group of an iron-loaded EDTA derivative (e.g., EDTA-2-aminoethyl-2-pyridyldisulfide (EPD)-Fe(II)) to covalently attach the targeting polypeptide and the iron-loaded EDTA derivative via a disulfide linkage. Further details regarding the attachment of chelating agents to proteins via disulfide linkages is found in, e.g., Hall and Fox (Methods (1999) 18:78-84).

Free Radical Generation

Upon contacting the target molecule with the free radical-generating system, the subject methods include generating free radicals from the free radical-generating system to deplete the target molecule in the sample. A sufficient amount of the free radicals are generated in close enough proximity to the target molecule such that the target molecule is depleted (e.g., undergoes cleavage and/or is modified such that target molecule adducts are formed, etc.) upon generation of the free radicals.

Generating free radicals may include contacting the free radical-generating system and/or an optional catalyst thereof with a free radical source. A particular source of free radicals may be selected based on the ability of the free radical-generating system to utilize the free radical source to generate free radicals and/or the desired type of free radicals to be generated. In certain embodiments, the free radicals are hydroxyl radicals (.OH) generated from a hydroxyl radical source. The hydroxyl radicals may be generated via a Fenton or Fenton-type reaction involving a divalent metal ion catalyst (e.g., Fe²⁺, Cu²⁺, Ni²⁺, Ca²⁺, or Zn²⁺), a peroxide (e.g., H₂O₂), and a reducing agent (e.g., ascorbate, mercaptopropionic acid (MPA), or the like).

Accordingly, in certain aspects, the subject methods employ a free radical-generating system that includes a catalyst-binding agent (e.g., a chelating agent) bound to a catalyst (e.g., a divalent metal ion catalyst such as Fe²⁺ or Cu²⁺), the catalyst-binding agent including a targeting molecule such as a targeting nucleic acid or targeting polypeptide that brings the free radical-generating system within close proximity to the target molecule. When such a free radical-generating system is employed, the free radical-generating system may be contacted with a peroxide (e.g., H₂O₂) to generate hydroxyl radicals at the location of the metal ion catalyst. When the target molecule is a nucleic acid (e.g., a ribosomal RNA such as a eukaryotic 28S, 26S, 25S, 18S, 5.8S, or 5S rRNA) bound by the free radical-generating system (via the targeting molecule), the generation of hydroxyl radicals results in cleavage of phosphodiester bonds in the target nucleic acid, the generation of target nucleic acid adducts, or the like, thereby depleting the target nucleic acid in the sample. Similarly, when the target polymer is a polypeptide, the polypeptide may be depleted as a result of free radical-induced cleavage of peptide bonds in the polypeptide, the generation of target polypeptide adducts, or the like, thereby depleting the target polypeptide in the sample.

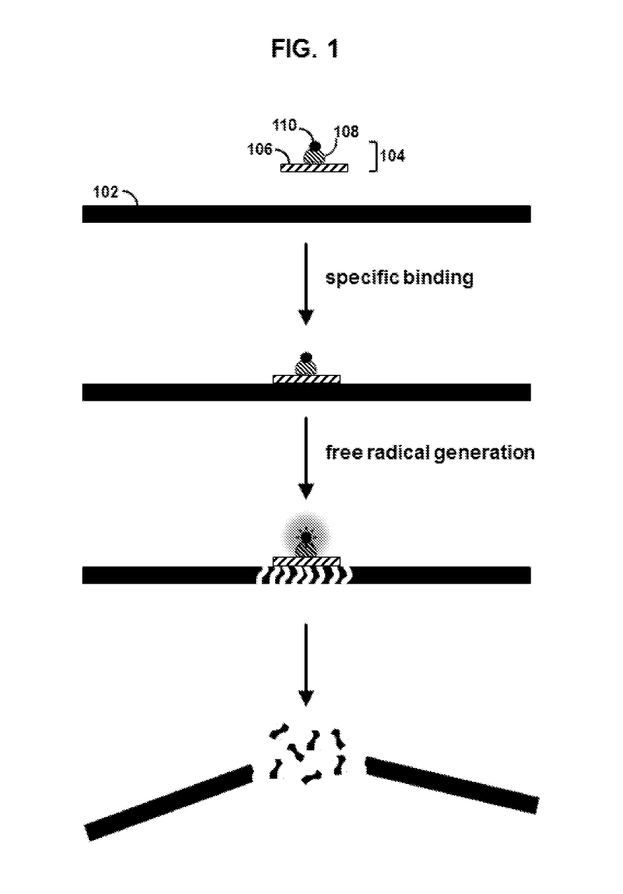

A method according to one embodiment of the present disclosure is schematically illustrated in FIG. 1. Free radical-generating system 104 is provided in a sample of interest that includes target molecule 102. In this example, free radical-generating system 104 includes targeting molecule 106, catalyst-binding agent 108 attached to targeting molecule 106, and catalyst 110 bound to catalyst-binding agent 108. Target molecule 102 is contacted with free radical-generating system 104 via specific binding of the targeting molecule to the target molecule. Upon contacting target molecule 102 with free radical-generating system 104 and contacting catalyst 110 with a free radical source, free radicals are generated from a reaction catalyzed by the catalyst within sufficient proximity of the target molecule to deplete the target molecule in the sample.

Suitable reaction conditions (e.g., reaction times, temperatures, reagent concentrations, and the like) for generating free radicals vary according to factors such as the free radical generating system employed. In certain aspects, when a catalyst such as iron or copper is used and the free radical source is hydrogen peroxide, the final hydrogen peroxide concentration in the reaction is from 0% to 1%. The temperature at which the free radical generation occurs may vary, and in some instances may be from 0° C. to 40° C.

According to certain embodiments, free radical generation occurs for a duration from 0.03 hours to 3 hours. In certain aspects, when the generation of free radicals has occurred for a desired duration, the subject methods further include terminating the generation of free radicals using a quenching agent. The quenching agent may be a chemical or non-chemical agent. In certain embodiments, the quenching agent is a chemical agent, e.g., thiourea. The quenching agent may be added to the reaction volume at any convenient final concentration to quench the reaction.

### Kits

Also provided by the present disclosure are kits useful for practicing the subject methods. The kits include a free radical-generating system configured to deplete a target molecule upon contacting the target molecule in the presence of a free radical source. In certain aspects, the free radical-generating system includes a catalyst and a catalyst-binding agent. In certain aspects, when the free radical-generating system includes catalyst and a catalyst-binding agent, the catalyst-binding agent includes a targeting molecule configured that binds to the target molecule.

Free radical-generating systems of the subject kits may include any of the components (e.g., including optional catalysts, catalyst-binding agents, targeting molecules, etc.) as described above in relation to the subject methods. In certain aspects, the subject kits include a catalyst bound by a catalyst binding agent. In other aspects, the kit includes the catalyst binding agent attached to a targeting molecule and a catalyst bound to the catalyst-binding agent.

According to certain embodiments, the kit includes free radical-generating system components configured to deplete a target nucleic acid. The target nucleic may be any target nucleic acid described above in relation to the subject methods. As just one example, the subject kits may include free radical-generating system components configured to deplete a ribosomal RNA (rRNA) from a sample of interest.

Reagents, such as nucleases (e.g., exonuclease I) and/or proteases, may be included in the subject kits for removal/inactivation of the free radical-generating system after a free radical generation step is complete. The kits may also include reagents for performing precipitation reactions and/or spin purification columns to remove fragmented target molecules and/or targeting complexes from the sample, e.g., after the free radical generation step is complete. According to certain aspects, the subject kits include a quenching agent (e.g., a chemical quenching agent) for terminating a free radical generation reaction once the reaction has occurred for a desired duration.

In certain embodiments, the kits include reagents for isolating the target molecule from a target molecule source. The reagents may be suitable for isolating target molecule samples from a variety of sources including single cells, cultured cells, tissues, organs, or organisms. The subject kits may include reagents for isolating the sample from a fixed tissue or organ, e.g., formalin-fixed, paraffin-embedded (FFPE) tissue. Such kits may include one or more deparaffinization agents, one or more agents suitable to decrosslink nucleic acids or proteins, and/or the like.

Components of the subject kits may be present in separate containers, or multiple components may be present in a single container. For example, components of the free radical-generating system may be present in separate containers or in a single container.

In addition to above-mentioned components, the subject kit may further include instructions for using the components of the kit to practice the subject methods. For example, the kit may include instructions for depleting the target molecule using a free radical-generating system as supplied with the kit. The kit may include instructions for generating the free radical-generating system, e.g., if the components of the free radical-generating system are provided in separate containers. The instructions for practicing the subject methods and/or generating the free radical-generating system are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Utility

The subject methods and kits find use in a variety of different applications where, e.g., it is desirable to deplete irrelevant and/or undesired molecules from a sample of interest. By depleting the irrelevant and/or undesired molecules, the complexity of the sample is reduced and the sample is enriched for molecules of interest. When the molecules of interest are nucleic acids, reduced complexity and enrichment of nucleic acids of interest may facilitate and/or improve the results of downstream applications such as nucleic acid amplification, nucleic acid sequencing, gene expression analysis (e.g., by array hybridization, quantitative RT-PCR, massively parallel sequencing, etc.), the preparation of pharmaceutical compositions in which a therapeutic nucleic acid of interest is to be included, and any other applications in which reduced sample complexity and enrichment of nucleic acids of interest is beneficial.

As just one particular example, the subject methods and kits find use in facilitating gene expression analysis in nucleic acid samples derived from a nucleic acid source (e.g., a formalin fixed, paraffin-embedded tissue sample) in which the integrity of nucleic acids of interest (e.g., mRNAs) is often compromised. For example, the poly-A tails of mRNAs in formalin fixed, paraffin-embedded tissue samples are often absent or degraded to an extent that such tails do not support oligo-dT priming. Accordingly, if the method for enriching the nucleic acid sample for mRNAs is amplification by oligo-dT priming, a substantial proportion of the mRNAs present in the initial sample will not be amplified. The subject methods constitute an approach for enriching nucleic acids of interest that are less sensitive to sample degradation, e.g., because the undesired nucleic acids may be depleted in a manner that utilizes internal sequences of the undesired nucleic acids, which are more likely to remain intact in degraded samples as compared to the poly-A tails of mRNAs.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of depleting a target molecule in a sample, the method comprising:
(a) contacting a target molecule with a free radical-generating system; and
(b) generating free radicals from the free radical-generating system to deplete the target molecule in the sample.

2. The method according to claim 1, wherein the free radical-generating system comprises a catalyst and a catalyst-binding agent.

3. The method according to claim 2, wherein the catalyst-binding agent comprises a targeting molecule that specifically binds to the target molecule.

4. The method according to claim 2, wherein the catalyst-binding agent is a chelating agent.

5. (canceled)

6. The method according to claim 2, wherein the catalyst is a metal.

7. The method according to claim 6, wherein the metal is selected from copper and iron.

8. The method according to claim 7, wherein the catalyst is iron and the catalyst-binding agent is EDTA.

9. The method according to claim 1, wherein the target molecule is a target nucleic acid.

10. The method according to claim 9, wherein the target nucleic acid is a ribonucleic acid (RNA).

11. (canceled)

12. The method according to claim 10, wherein the RNA is an rRNA.

13. The method according to claim 9, wherein the target nucleic acid is a deoxyribonucleic acid (DNA).

14. The method according to claim 9, wherein the targeting molecule is selected from the group consisting of: a nucleic acid that specifically hybridizes to the target nucleic acid, a polypeptide, an intercalating agent, and an aptamer.

15. The method according to claim 14, wherein the targeting molecule is a nucleic acid that specifically hybridizes to the target nucleic acid.

16. (canceled)

17. The method according to claim 1, wherein the target polymer is a target polypeptide.

18. (canceled)

19. The method according to claim 2, wherein generating free radicals from the free radical-generating system comprises contacting the catalyst with a free radical source.

20. The method according to claim 19, wherein the free radical source is a hydroxyl radical source.

21. The method according to claim 20, wherein the hydroxyl radical source is hydrogen peroxide (H₂O₂).

22. The method according to claim 1, wherein after generating free radicals from the free radical-generating system, the method comprises terminating the generation of free radicals using a quenching agent.

23. The method according to claim 22, wherein the quenching agent is a chemical agent.

24. The method according to claim 23, wherein the chemical agent is thiourea.

25. A kit comprising:
a free radical-generating system configured to deplete a target molecule upon contacting the target molecule in the presence of a free radical source.

26-51. (canceled)
